(19) United States
(12) Patent Application Publication
(10) Pub. No.: US 2009/0023757 A1
(43) Pub. Date: Jan. 22, 2009

(54) PROCESS FOR PREPARING BICYCLIC COMPOUNDS

(76) Inventors: Daniele Andreotti, Verona (IT); Sergio Bacchi, Verona (IT); Monica Delpogetto, Verona (IT); Simone Guelfi, Verona (IT); Alcide Perboni, Verona (IT); Arianna Ribecai, Verona (IT); Simone Spada, Verona (IT); Paolo Stabile, Verona (IT); Marsia Tampieri, Verona (IT)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 11/910,990

(22) PCT Filed: Apr. 6, 2006
(86) PCT No.: PCT/EP2006/003480

§ 371 Date: Feb. 26, 2008

(30) Foreign Application Priority Data
Apr. 8, 2005 (GB) 0507195.6

(51) Int. Cl. A61K 31/519; C07D 471/04; A61P 1/00; C07D 207/14

(52) U.S. Cl. 514/265.1; 546/113; 548/559

(57) ABSTRACT

The present invention relates to a novel process for preparing compounds of formula (IA), which are potent and specific antagonists of corticotropin-releasing factor (CRF) receptors, from intermediate compounds of formula (I), by a coupling reaction catalysed by copper.

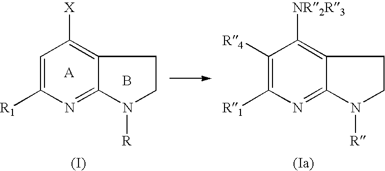

The present invention relates to a novel process and an intermediate compound, useful for preparing key intermediates in the synthesis of various bicyclic compounds, which are potent and specific antagonists of corticotropin-releasing factor (CRF) receptors.

The first corticotropin-releasing factor (CRF) was isolated from ovine hypothalami and identified as a 41-amino acid peptide (Vale et al., Science 213: 1394-1397,1981).

CRF has been found to produce profound alterations in endocrine, nervous and immune system function. CRF is believed to be the major physiological regulator of the basal and stress-release of adrenocorticotropic hormone (“ACTH”), Bendorphin and other proopiomelanocortin (“POMC”)-derived peptides from the anterior pituitary (Vale et al., Science 213: 1394-1397,1981).

In addition to its role in stimulating the production of ACTH and POMC, CRF appears to be one of the pivotal central nervous system neurotransmitters and plays a crucial role in integrating the body's overall response to stress.

Administration of CRF directly to the brain elicits behavioral, physiological and endocrine responses identical to those observed for an animal exposed to a stressful environment. Accordingly, clinical data suggests that CRF receptor antagonists may represent novel antidepressant and/or anxiolytic drugs that may be useful in the treatment of the neuropsychiatric disorders manifesting hypersecretion of CRF.

The present invention relates to a novel process for preparing bicyclic CRF antagonists of formula (IA), as disclosed in WO 03/008412, starting from key intermediates of general formula (I),

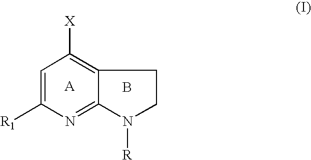

In compounds of formula (I), R and R₁, are defined as in WO 03/008412, i.e.:
R is aryl or heteroaryl, each of which may be substituted by 1 to 4 groups selected from:
halogen, C1-C6 alkyl, C1-C6 alkoxy, halo C1-C6 alkyl, C2-C6 alkenyl, C2-C6 alkynyl, halo C1-C6 alkoxy, —C(O)R₅, nitro, —NR₆R₇, cyano, and a group R₈;
R₁ is hydrogen, C1-C6 alkyl, C2-C6 alkenyl, C2-C6 alkynyl, halo C1-C6 alkyl, halo C1-C6 alkoxy, halogen, NR₆R₇or cyano;
R₅ is a C1-C4 alkyl, —OR₆ or —NR₆R₇;
R₆ is hydrogen or C1-C6 alkyl;
R₇ is hydrogen or C1-C6 alkyl;
R₈ is a 5-6 membered heterocycle, which may be saturated or may contain one to three double bonds, and which may be substituted by 1 or more R₁₁ groups;
R₉ is a C1-C6 alkyl that may be substituted by one or more groups selected from: C3-C7 cycloalkyl, C1-C6 alkoxy, haloC1-C6 alkoxy, hydroxy, haloC1-C6 alkyl;
R₁₁ is C3-C7 cycloalkyl, C1-C6 alkyl, C1-C6 alkoxy, halo C1-C6 alkyl, C2-C6 alkenyl, C2-C6 alkynyl, halo C1-C6 alkoxy, hydroxy, halogen, nitro, cyano, or C(O)NR₆R₇;
X is halogen.

In one aspect the present invention provides a process useful for the preparation of compounds of formula (IA), as disclosed in WO 03/008412:

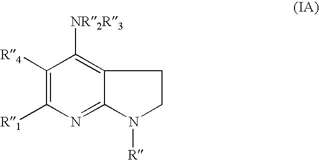

wherein
R″ corresponds to R;
R″₁ corresponds to R₁;
R₂ is hydrogen, C3-C7 cycloalkyl, or a group R₉;
R₃ is C3-C7 cycloalkyl, or a group R₉; or
R₂ and R₃ together with N form a 5-14 membered heterocycle, which may be substituted by 1 to 3 R₁₀ groups;

R″₄ is hydrogen;

R″₅ corresponds to R₅;

R″₆ corresponds to R₆;

R″₇ corresponds to R₇;

R″₈ corresponds to R₈;

R″₉ corresponds to R₉;

R₁₀ is a group R₈, C3-C7 cycloalkyl, C1-C6 alkyl, C1-C6 alkoxy, halo C1-C6 alkyl, C2-C6 alkenyl, C2-C6 alkynyl, halo C1-C6 alkoxy, hydroxy, halogen, nitro, cyano, C(O)NR₆R₇, phenyl which may be substituted by 1 to 4 R₁₁ groups;

R″₁₁ corresponds to R₁₁.

In another aspect the present invention provides a process useful for the preparation of compounds of formula (IIA):

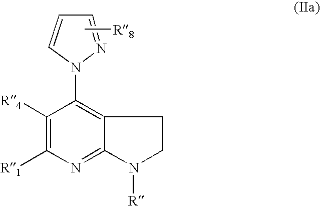

which correspond to compounds of formula (IA) where R″₂ and R″₃ form a pyrazole ring and R″, R₁″ R″₄, R″₈ are defined as above.

The term C1-C6 alkyl as used herein as a group or a part of the group refers to a linear or branched alkyl group containing from 1 to 6 carbon atoms; examples of such groups include methyl, ethyl, propyl, isopropyl, n-butyl, isobutyl, tert butyl, pentyl or hexyl.

The term C3-C7 cycloalkyl group means a non aromatic monocyclic hydrocarbon ring of 3 to 7 carbon atom; examples of such groups include cyclopropyl, cyclobutyl, cyclopentyl, cyclohexyl or cycloheptyl; while unsaturated cycloalkyls include cyclopentenyl and cyclohexenyl, and the like.

The term halogen refers to a fluorine, chlorine, bromine or iodine atom.

The term halo C1-C6 alkyl, or halo C1-C2 alkyl means an alkyl group having one or more carbon atoms and wherein at least one hydrogen atom is replaced with halogen such as for example a trifluoromethyl group and the like.

The term C1-C6 thioalkyl may be a linear or a branched chain thioalkyl group, for example thiomethyl, thioethyl, thiopropyl, thioisopropyl, thiobutyl, thiosec-butyl, thiotert-butyl and the like.

The term C2-C6 alkenyl defines straight or branched chain hydrocarbon radicals containing one or more double bond and having from 2 to 6 carbon atoms; examples of such groups include ethenyl, 2-propenyl, 3-butenyl, 2-butenyl, 2-pentenyl, 3-pentenyl, 3-methyl-2-butenyl or 3-hexenyl and the like.

The term C1-C6 alkoxy group may be a linear or a branched chain alkoxy group; examples of such groups include methoxy, ethoxy, propoxy, prop-2-oxy, butoxy, but-2-oxy or methylprop-2-oxy and the like.

The term halo C1-C6 alkoxy group may be a C1-C6 alkoxy group as defined before substituted with at least one halogen; examples of such groups include OCHF₂ or OCF₃.

The term C2-C6 alkynyl defines straight or branched chain hydrocarbon radicals containing one or more triple bond and having from 2 to 6 carbon atoms including acetylenyl, propynyl, 1-butynyl, 1-pentynyl, 3-methyl-1-butynyl and the like.

The term aryl means an aromatic carbocyclic moiety such as phenyl, biphenyl or naphthyl.

The term heteroaryl means an aromatic heterocycle ring of 5 to 10 members and having at least one heteroatom selected from nitrogen, oxygen and sulfur, and containing at least 1 carbon atom, including both mono-and bicyclic ring systems.

Representative heteroaryls include (but are not limited to) furyl, benzofuranyl, thiophenyl, benzothiophenyl, pyrrolyl, indolyl, isoindolyl, azaindolyl, pyridyl, quinolinyl, isoquinolinyl, oxazolyl, isooxazolyl, benzoxazolyl, pyrazolyl, imidazolyl, benzimidazolyl, thiazolyl, benzothiazolyl, isothiazolyl, pyridazinyl, pyrimidinyl, pyrazinyl, triazinyl, cinnolinyl, phthalazinyl, triazolyl, tetrazolyl, quinazolinyl, and benzodioxolyl.

The term 5-6 membered heterocycle means, according to the above definition, a 5-6 monocyclic heterocyclic ring which is either saturated, unsaturated or aromatic, and which contains from 1 to 4 heteroatoms independently selected from nitrogen, oxygen and sulfur, and wherein the nitrogen and sulfur heteroatoms may be optionally oxidized, and the nitrogen heteroatom may be optionally quaternized. Heterocycles include heteroaryls as defined above. The heterocycle may be attached via any heteroatom or carbon atom. Thus, the term includes (but is not limited to) morpholinyl, pyridinyl, pyrazinyl, pyrazolyl, thiazolyl, triazolyl, imidazolyl, oxadiazolyl, oxazolyl, isoxazolyl, pyrrolidinonyl, pyrrolidinyl, piperidinyl, hydantoinyl, valerolactamyl, oxiranyl, oxetanyl, tetrahydrofuranyl, tetrahydropyranyl, tetrahydropyridinyl, tetrahydropyrimidinyl, tetrahydrothiophenyl, tetrahydrothiopyranyl, and the like.

In one aspect the present invention provides a process for preparing compounds of formula (IA) starting from compounds of formula (I), by a coupling reaction catalysed by copper

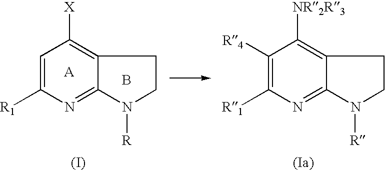

In one embodiment of the present invention the coupling reaction, similar to the Goldberg reaction, may be performed according to the following procedure.

A solution of a suitable copper catalyst selected in the group consisting from: CuI, CuBr, Cu₂Br, Cu(AcO)₂, Cu₂O; and a suitable ligand selected in the group consisting from: cis- or trans-N,N′-dimethyl-1,2-cyclohexanediamine, a mixture of cis- and trans-N,N′-dimethyl-1,2-cyclohexanediamine, cis- or trans-1,2-cyclohexanediamine, a mixture of cis- and trans-1,2-cyclohexanediamine, N,N′-dimethyl-1,2-diaminoethane, NN,N′N′-tetramethyl-1,2-diaminoethane, ethanolamine, 1,10-phenantroline, triphenylphosphine, BINAP, Acac; is prepared in a suitable solvent selected among polar aprotic solvents as defined above, or toluene, dioxane, 1,2-bis(methyloxy)ethane.

Then an inorganic or organic base as defined above is added followed by the reactive derivative of the upper residue (group —NR′″₂R′″₃ in compounds of formula (I)) and the suitable intermediate compound (I). The resulting mixture is then kept at a temperature ranging from 80° to 150° C. for 4-48 hr.

The mixture is then cooled at the end and worked as usual in order to provide a two layers mixture. The organic layer is constitued by a suitable organic solvent as described above. A suitable solvent may be added for improving the precipitation.

In one aspect the present invention provides a process for preparing the following compounds:

3-Methyl-4-[6-methyl-4-(3-thiazol-2-yl-pyrazol-1-yl)-2,3-dihydro-1H-pyrrolo[2,3-b]pyridin-1-yl]-benzonitrile;

1-(2,4-Bis-trifluoromethyl-phenyl)-6-methyl-4-(3-thiazol-2-yl-pyrazol-1-yl)-2,3-dihydro-1H-pyrrolo[2,3-b]pyridine;

4-[6-Methyl-4-(3-thiazol-2-yl-pyrazol-1-yl)-2,3-dihydro-1H-pyrrolo[2,3-b]pyridin-1-yl]-3-trifluoromethyl-benzonitrile;

6-Methyl-1-(2-methyl-4-trifluoromethoxy-phenyl)-4-(3-thiazol-2-yl-pyrazol-1-yl)-2,3-dihydro-1H-pyrrolo[2,3-b]pyridine;

1-(4-Methoxy-2-methyl-phenyl)-6-methyl-4-(3-thiazol-2-yl-pyrazol-1-yl)-2,3-dihydro-1H-pyrrolo[2,3-b]pyridine;

1-(2,4-Bis-trifluoromethyl-phenyl)-6-methyl-4-(3-morpholin-4-yl-pyrazol-1-yl)-2,3-dihydro-1H-pyrrolo[2,3-b]pyridine;

1-(2,4-Bis-trifluoromethyl-phenyl)-6-methyl-4-(3-pyridin-2-yl-pyrazol-1-yl)-2,3-dihydro-1H-pyrrolo[2,3-b]pyridine;

4-[1,3′]Bipyrazolyl-1′-yl-1-(2,4-bis-trifluoromethyl-phenyl)-6-methyl-2,3-dihydro-1H-pyrrolo[2,3-b]pyridine.

In one aspect, the present invention provides the preparation of 6-methyl-1-[2-methyl-4-(methyloxy)phenyl]-4-[3-(1,3-thiazol-2-yl)-1-H-pyrazol-1-yl]-2,3-dihydro-1H-pyrrolo[2,3-b]-pyridine which is reported in the Experimental section as illustrative of the procedure object of the present invention.

The other compounds mentioned above may be easily prepared according to the procedure described for 6-methyl-1-[2-methyl-4-(methyloxy)phenyl]-4-[3-(1,3-thiazol-2-yl)-1-H-pyrazol-1-yl]-2,3-dihydro-1H-pyrrolo[2,3-b]pyridine.

In another aspect the present invention provides the CRF antagonist compound of formula (IX), 6-methyl-1-[2-methyl-4-(methyloxy)phenyl]-4-[5-(1,3-thiazol-2-yl)-1H-pyrazol-1-yl]-2,3-dihydro-1H-pyrrolo[2,3-b]pyridine, which is a side product formed during the on large scale preparation of 6-methyl-1-[2-methyl-4-(methyloxy)phenyl]-4-[3-(1,3-thiazol-2-yl)-1-H-pyrazol-1-yl]-2,3-dihydro-1H-pyrrolo[2,3-b]pyridine.

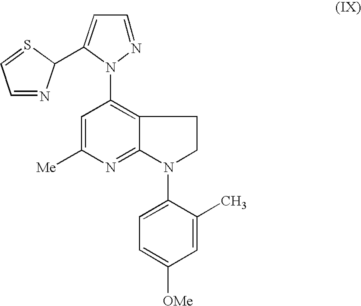

The compound (IX) is a novel compound and it is another embodiment of the present invention. The compound (IX) has been tested by using the homogeneous technique of scintillation proximity (SPA). The ligand binds to recombinant membrane preparation expressing the CRF receptors which in turn bind to wheatgerm agglutinin coated SPA beads. In the Experimental Part will be disclosed the details of the experiments. The compound has a Ki less than 0.1 μm.

Compounds of formula (I) may be prepared as disclosed in WO 04/094420, WO 03/008412 and WO 04/062665. Alternatively they may be prepared from compounds of formula (VII).

The compounds of formula (VII) are intermediates in the process for the preparation of compounds of formula (I), according to the following Scheme 1:

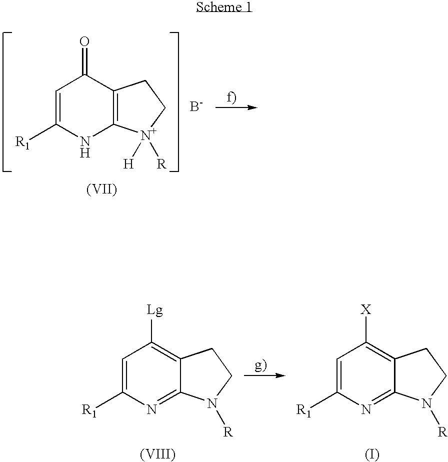

wherein R, R_{1,} and X are defined as above, and Lg is a leaving group selected among the reactive derivatives of an alkylsulphonic acid and
step f stands for the formation of a reactive derivative of the hydroxy pyridine of compounds (VII);
step g stands for nucleophilic displacement of the reactive derivative of compounds (VIII) to give the halogenated compounds (I).

Step f stands for the formation of a reactive derivative (i.e. a leaving group, Lg) of the hydroxy pyridine. The leaving group may be a reactive derivative of an alkylsulphonic acid, which includes but it is not limited to mesylate, tosylate, triflate.

To a suspension of intermediate compound (VII) in a suitable solvent which includes, but it is not limited to, chlorinated solvents (e.g. dichloromethane), an inorganic base in aqueous solution is added in order to provide the corresponding salt.

The suitable inorganic base may be selected from the group consisting of: sodium carbonate, sodium hydrogen carbonate, potassium carbonate, potassium hydrogen carbonate, sodium hydroxyde, potassium hydroxyde.

The salt so formed may be separated and then an organic amine is added at R.T. under N₂. In one embodiment of the present invention the organic amine may be pyridine or triethylamine.

The mixture is then cooled down to low temperature (below −10° C.) and triflic anhydride or methanesulfonic anhydride or methanesulfonyl chloride is added carefully. The reaction mixture is then usually worked-up.

In another embodiment of the present invention the solution may be added with pure seeds of the desired intermediate compound (VIII), previously prepared.

Step g stands for nucleophilic displacement of the leaving group of compounds (VIII) to give the compounds of formula (I).

In one embodiment of the present invention X may be Iodine.

In another embodiment X may be Bromine.

To a solution of intermediate compounds (VIII) in a suitable solvent which includes, but it is not limited to, a polar aprotic solvent selected in the group consisting of: dimethylformamide (DMF), dimethylsulfoxide (DMSO), N-methylpyrrolidinone (NMP), acetonitrile, a linear or branched C1-C6 alcoholic solvent or an apolar solvents, an organic acid selected in the group consisting from: methansulfonic acid, acetic acid, p-toluenesulfonic acid, trifluoroacetic acid, fumaric acid was added, followed by the addition of a halide salt with alkaline ions which includes: LiCl, LiBr, LiI, NaCl, NaBr, NaI, KCl, KBr, or KI.

The resulting mixture is usually kept at a temperature ranging from 50 to 120° C. for 2-24 hr. At the end the reaction mixture is worked-up as usual in order to provide a two layers mixture. The organic layer is usually constitued by a suitable organic solvent such as an etheral or ester solvent, as defined above.

The crude product may be used as such in the next step for the formation of the bicylic CRF antagonists which will be defined in the following

Compounds of formula (VI) may be prepared as disclosed in WO 04/062665 and WO 04/094420.

Compounds of formula (VI) may exist in the tautomeric form.

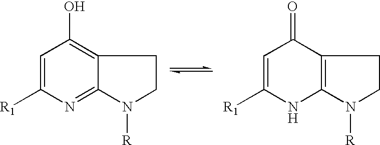

A process for the preparation of compounds (IV) is one embodiment of the present invention, starting from compounds of formula (II) and comprising the following steps according to Scheme 2:

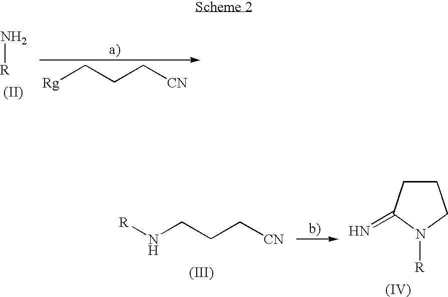

wherein R is defined as above, Rg is a reactive group selected from: halogen, reactive derivative of an alkylsulphonic acid, and
step a stands for alkylation of the suitable aryl or heteroayl amine of formula (II) with a reactive derivative of butyrronitrile in presence of a base by heating;
step b stands for the formation of the pyrrolidinone moiety of compounds (IV) which will form the cycle B present in the final compounds (I), by cyclisation of compounds (Ill), acid catalised and by heating to give the desired compounds (IV).

The starting R—NH₂ may be a compound generally already known in literature. If not, it may be prepared using classical approach known to the skilled person.

Step a stands for alkylation of the suitable aryl or heteroayl amine of formula (II) with a reactive derivative of butyrronitrile in presence of a base by heating.

The suitable aryl or heteroaryl amine is dissolved in a proper solvent which includes, but it is not limited to, a tertiary C1-C6 dialkylamine.

In one embodiment of the present invention the tertiary C1-C6 dialkylamine may be trietylamine or diisopropylamine together, if necessary, with a polar aprotic solvent selected in the group consisting of: dimethylformamide (DMF), dimethylsulfoxide (DMSO), N-methylpyrrolidinone (NMP), acetonitrile.

The reaction is usually conducted at a temperature comprised in the range 100-150° C.

In one embodiment of the present invention the reactive derivative of butyrronitrile is an halogen derivative. In a further embodiment the halogen may be Cl or Br.

The reactive derivative is added dropwise under N₂. The reaction mixture is then stirred for 2-6 hr. The mixture is then cooled down to R.T. and diluted with a suitable solvent which includes, but it is not limited to, linear, branched or cyclic C1-C6 dialkylether.

In one embodiment of the present invention the solvent may be selected from the group consisting of: methyl-t-butyl ether, dietylether, tetrahydrofuran, or dioxane.

The reaction mixture is then worked up as usual and at the end a suitable co-solvent is added. A suitable co-solvent may be selected in the group of C1-C10 cyclic alcanes.

In one embodiment of the present invention the co-solvent may be cyclohexane.

The crude product may be used as such in the next step.

Step b stands for the formation of the pyrrolidinone moiety of compounds (I) which will form the cycle B present in the final compounds (I), by cyclisation of compounds (III).

To a suspension of intermediate compounds (III) in a suitable solvent, which includes, but it is not limited to, a linear or branched C1-C6 alcoholic solvent or a C1-C10 aromatic solvent or a linear, branched or cyclic C1-C6 dialkylether.

In one embodiment of the present invention the alcoholic solvent may be iso-propanol; the aromatic solvent may be toluene and the etheral solvent may be tetrahydrofuran (THF).

Then 1.5 eq. of an acid are added at R.T. under N₂.

The most suitable acid may be selected among the organic acid or inorganic acids common to the skilled person.

Organic acids include, but are not limited to: acetic acid, malic acid, maleic acid, fumaric acid, lactic acid, tartaric acid, citric acid, formic acid, gluconic acid, succinic acid, piruvic acid, oxalic acid, oxaloacetic acid, trifluoroacetic acid, benzoic acid, methansulphonic acid, ethanesulphonic acid, benzenesulphonic acid, p-toluensulphonic acid, methanesulphonic acid and isethionic acid.

Inorganic acids include, but are not limited to: hydrochloric acid, hydrobromic acid, hydroiodic acid, sulphoric acid, nitric acid, phosphoric acid, hydrogen phosphoric acid.

In one embodiment of the present invention the organic acid may be p-toluenesulfonic acid or methanesulfonic acid and the inorganic acid may be hydrochloric acid (HCl).

The mixture is then usually heated to reflux for 4-8 hr, and at the end worked as usual in order to provide a two layers mixture. The organic layer is usually constitued by a suitable organic solvent which includes, but it is not limited to, chlorinated solvents or esters of organic acids.

In one embodiment of the present invention the chlorinated solvent may be dichloromethane and the ester of organic acid may be ethylacetate.

The crude product may be used as such in the next step.

In one aspect of the present invention step a and step b may be performed continuously without isolating intermediate (III), according to the following Scheme 3, in order to produce compounds of formula (IVB), which can be used as compounds (IV) after treatment in basic conditions.

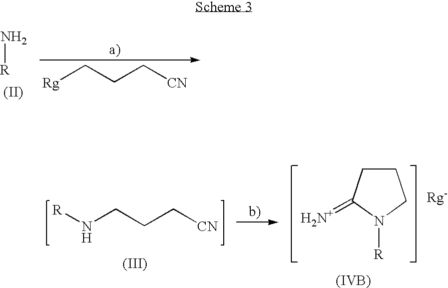

Compounds (IV) may be isolated as a suitable salt, for example hydrobromide, depending of the type of reactive butyrronitrile used in step b). Then bromobutyrronitrile will be used for obtaining compounds (IVB) as hydrobromide

The preparation of 1-[2-methyl-4-(trifluoromethyloxy)phenyl]-2-pyrrolidinimine hydrobromide included in the Experimental Section is an illustration of this alternative way of performing the process of the present invention.

A process for the preparation of compounds (VII) is another embodiment of the present invention, starting from compounds of formula (IV) and comprising the following steps:

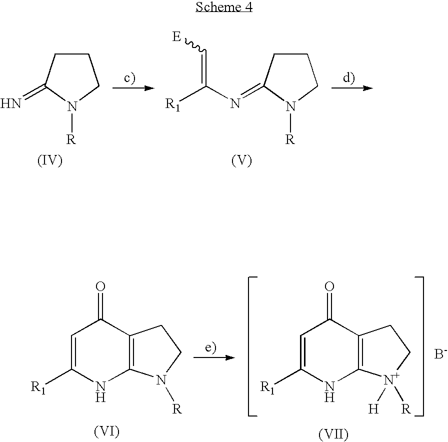

wherein R and R_{1,} are defined as above, and
step c stands for a Michael addition of compounds (IV) to a butynoate derivative by heating;
step d stands for cyclisation in basic conditions to give the aromatic compounds (VI);
step e stands for salt formation by addition of the suitable acid to the compounds (VI).

Compounds (IV) may be replaced in Scheme 4 by compounds (IVB) providing an initial step c′ of basic treatment in a suitable base as illustrated in the following Scheme 5.

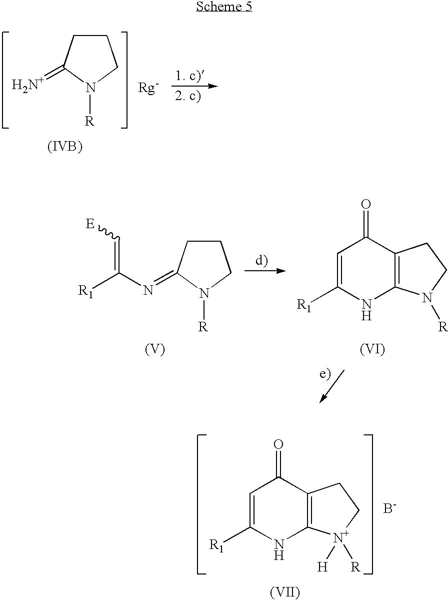

Step c stands for a Michael addition of intermediate compounds (IV) to a suitable butynoate derivative.

To a solution of intermediate compounds (IV) in a suitable solvent which includes but it is not limited to, an etheral solvent, a polar aprotic solvent or an alcoholic solvent as defined above, 1.0-1.5 eq of an ester derivative of 2-butynoate is added at R.T. under N₂.

In one embodiment of the present invention the ester derivative of 2-butynoate may be ethyl 2-butynoate.

The mixture was heated to reflux and kept for 2-20 hr before allowing cooling down to R.T. The reaction mixture was then evaporated to dryness. The crude oil may be used as such in the next step.

Step d stands for cyclisation in basic conditions of the intermediate compounds (V) to give the aromatic compounds (VI). To a solution of the intermediate compounds (V) in a suitable solvent selected among etheral solvents, alcoholic solvents or polar aprotics solvents as defined above, a suitable base selected in the group consisting from: potassium t-butoxide, lithium hexamethyidisilazane, diazabicyclo[2.2.2]octane, 1,8-diazabicyclo[5.4.0]undecen-7-ene, sodium hydride; is added at R.T. under N₂.

The reaction mixture is then generally heated to reflux and stirred for 2-14 hr and at the end worked as usual in order to provide a two layers mixture. The organic layer is usually constitued by a suitable organic solvent which includes, but it is not limited to, chlorinated solvents.

In one embodiment of the present invention the chlorinated solvent may be dichloromethane.

The crude product may be used as such in the next step.

Step e stands for the formation of compounds (VII) by addition of the suitable acid to the intermediate compounds (VI).

A compound (VI) is dissolved in a suitable solvent which includes, but it is not limited to, a linear, branched or cyclic C1-C6 dialkylether, a linear or branched aliphatic C1-C6 ketonic solvent. The solution is then treated with a suitable inorganic acid.

In one embodiment of the present invention the ketonic solvent may be acetone or 2-butanone, the etheral solvent may be tethrahydrofurane (THF) and the acid may be a sulphonic acid. In a further embodiment the sulphonic acid may be p-toluensulphonic acid or methanesulphonic acid.

In another embodiment the solution may be added with pure seeds of the desired intermediate compound (VII), previously prepared.

After 2-10 hr the suspension is filtered and the cake washed with another solvent.

The collected solid is then dried in the usual way.

The formation of compounds (VII) improves the process management as far as regards the purification procedures. In fact, by the introduction of these salts formation it is now possible to have reasonable clean intermediates without using chromatographic procedures. In addition isolation of such intermediates allows a better control over the impurity profile in the next steps.

The compound of the invention is useful in the treatment of central nervous system disorders where CRF receptors are involved. In particular in the treatment or prevention of major depressive disorders including bipolar depression, unipolar depression, single or recurrent major depressive episodes with or without psychotic features, catatonic features, melancholic features, atypical features or postpartum onset, the treatment of anxiety and the treatment of panic disorders. Other mood disorders encompassed within the term major depressive disorders include dysthymic disorder with early or late onset and with or without atypical features, neurotic depression, post traumatic stress disorders, post operative stress and social phobia; dementia of the Alzheimer's type, with early or late onset, with depressed mood; vascular dementia with depressed mood; mood disorders induced by alcohol, amphetamines, cocaine, hallucinogens, inhalants, opioids, phencyclidine, sedatives, hypnotics, anxiolytics and other substances; schizoaffective disorder of the depressed type; and adjustment disorder with depressed mood. Major depressive disorders may also result from a general medical condition including, but not limited to, myocardial infarction, diabetes, miscarriage or abortion, etc.

The compound of the invention is also useful in the treatment or prevention of schizophrenic disorders including: paranoid schizophrenia, disorganised schizophrenia, catatonic schizophrenia, undifferentiated schizophrenia, residual schizophrenia.

The compound of the invention is useful as analgesic. In particular it is useful in the treatment of traumatic pain such as postoperative pain; traumatic avulsion pain such as brachial plexus; chronic pain such as arthritic pain such as occurring in osteo-, rheumatoid or psoriatic arthritis; neuropathic pain such as post-herpetic neuralgia, trigeminal neuralgia, segmental or intercostal neuralgia, fibromyalgia, causalgia, peripheral neuropathy, diabetic neuropathy, chemotherapy-induced neuropathy, AIDS related neuropathy, occipital neuralgia, geniculate neuralgia, glossopharyngeal neuralgia, reflex sympathetic dystrophy, phantom limb pain; various forms of headache such as migraine, acute or chronic tension headache, temporomandibular pain, maxillary sinus pain, cluster headache; odontalgia; cancer pain; pain of visceral origin; gastrointestinal pain; nerve entrapment pain; sport's injury pain; dysmennorrhoea; menstrual pain; meningitis; arachnoiditis; musculoskeletal pain; low back pain e.g. spinal stenosis; prolapsed disc; sciatica; angina; ankylosing spondyolitis; gout; burns; scar pain; itch; and thalamic pain such as post stroke thalamic pain.

The compound of the invention is also useful for the treatment of dysfunction of appetite and food intake and in circumstances such as anorexia, anorexia nervosa and bulimia.

The compound of the invention is also useful in the treatment of sleep disorders including dysomnia, insomnia, sleep apnea, narcolepsy, and circadian rhythmic disorders.

The compound of the invention is also useful in the treatment or prevention of cognitive disorders. Cognitive disorders include dementia, amnestic disorders and cognitive disorders not otherwise specified.

Furthermore the compound of the invention is useful as memory and/or cognition enhancers in healthy humans with no cognitive and/or memory deficit.

The compound of the invention is also useful in the treatment of tolerance to and dependence on a number of substances. For example, it is useful in the treatment of dependence on nicotine, alcohol, caffeine, phencyclidine (phencyclidine like compounds), or in the treatment of tolerance to and dependence on opiates (e.g. cannabis, heroin, morphine) or benzodiazepines; in the treatment of cocaine, sedative ipnotic, amphetamine or amphetamine-related drugs (e.g. dextroamphetamine, methylamphetamine) addiction or a combination thereof.

The compound of the invention is also useful as anti-inflammatory agents. In particular they are useful in the treatment of inflammation in asthma, influenza, chronic bronchitis and rheumatoid arthritis; in the treatment of inflammatory diseases of the gastrointestinal tract such as Crohn's disease, ulcerative colitis, postoperative gastric ileus (POI), inflammatory bowel disease (IBD) and non-steroidal anti-inflammatory drug induced damage; inflammatory diseases of the skin such as herpes and eczema; inflammatory diseases of the bladder such as cystitis and urge incontinence; and eye and dental inflammation.

The compound of the invention is useful in the treatment of allergic disorders, in particular allergic disorders of the skin such as urticaria, and allergic disorders of the airways such as rhinitis.

The compound of the invention is also useful in the treatment of emesis, i.e. nausea, retching and vomiting. Emesis includes acute emesis, delayed emesis and anticipatory emesis. The compound of the invention is useful in the treatment of emesis however induced. For example, emesis may be induced by drugs such as cancer chemotherapeutic agents such as alkylating agents, e.g. cyclophosphamide, carmustine, lomustine and chlorambucil; cytotoxic antibiotics, e.g. dactinomycin, doxorubicin, mitomycin-C and bleomycin; anti-metabolites, e.g. cytarabine, methotrexate and 5-fluorouracil; vinca alkaloids, e.g. etoposide, vinblastine and vincristine; and others such as cisplatin, dacarbazine, procarbazine and hydroxyurea; and combinations thereof; radiation sickness; radiation therapy, e.g. irradiation of the thorax or abdomen, such as in the treatment of cancer; poisons; toxins such as toxins caused by metabolic disorders or by infection, e.g. gastritis, or released during bacterial or viral gastrointestinal infection; pregnancy; vestibular disorders, such as motion sickness, vertigo, dizziness and Meniere's disease; post-operative sickness; gastrointestinal obstruction; reduced gastrointestinal motility; visceral pain, e.g. myocardial infarction or peritonitis; migraine; increased intercranial pressure; decreased intercranial pressure (e.g. altitude sickness); opioid analgesics, such as morphine; and gastro-oesophageal reflux disease, acid indigestion, over-indulgence of food or drink, acid stomach, sour stomach, waterbrash/regurgitation, heartburn, such as episodic heartburn, nocturnal heartburn, and meal-induced heartburn and dyspepsia.

The compound of the invention is of particular use in the treatment of gastrointestinal disorders such as irritable bowel syndrome (IBS); skin disorders such as psoriasis, pruritis and sunburn; vasospastic diseases such as angina, vascular headache and Reynaud's disease; cerebral ischeamia such as cerebral vasospasm following subarachnoid haemorrhage; fibrosing and collagen diseases such as scleroderma and eosinophilic fascioliasis; disorders related to immune enhancement or suppression such as systemic lupus erythematosus and rheumatic diseases such as fibrositis; and cough.

The compound of the invention is useful for the treatment of neurotoxic injury which follows cerebral stroke, thromboembolic stroke, hemorrhagic stroke, cerebral ischemia, cerebral vasospam, hypoglycemia, hypoxia, anoxia, perinatal asphyxia cardiac arrest.

The invention therefore provides the compound of formula (IX) or a pharmaceutically acceptable salt or solvate thereof for use in therapy, in particular in human medicine.

There is also provided as a further aspect of the invention the use of the compound of formula (IX) or a pharmaceutically acceptable salt or solvate thereof in the preparation of a medicament for use in the treatment of conditions mediated by CRF.

In an alternative or further aspect there is provided a method for the treatment of a mammal, including man, in particular in the treatment of condition mediated by CRF, comprising administration of an effective amount of a compound of formula (I) or a pharmaceutically acceptable salt or a solvate thereof.

While it is possible that, for use in therapy, the compound of the present invention may be administered as the raw chemical, it is preferable to present the active ingredient as a pharmaceutical formulation e. g. when the agent is in admixture with a suitable pharmaceutical excipient, diluent or carrier selected with regard to the intended route of administration and standard pharmaceutical practice.

In a further aspect, the invention provides a pharmaceutical composition comprising the compound of the invention or a pharmaceutically acceptable derivative thereof in association with a pharmaceutically acceptable carrier and/or excipient. The carrier and/or excipient must be “acceptable” in the sense of being compatible with the other ingredients of the formulation and not deletrious to the recipient thereof.

Accordingly, the present invention further provides a pharmaceutical formulation comprising the compound of the invention or a pharmaceutically acceptable derivative thereof, in association with a pharmaceutically acceptable carrier and/or excipient. The carrier and/or excipient must be “acceptable” in the sense of being compatible with the other ingredients of the formulation and not deletrious to the receipient thereof.

There is further provided by the present invention a process of preparing a pharmaceutical composition, which process comprises mixing at least one compound of the invention or a pharmaceutically acceptable derivative thereof, together with a pharmaceutically acceptable carrier and/or excipient.

The pharmaceutical compositions may be for human or animal usage in human and veterinary medicine and will typically comprise any one or more of a pharmaceutically acceptable diluent, carrier or excipient. Acceptable carriers or diluents for therapeutic use are well known in the pharmaceutical art, and are described, for example, in Remington's Pharmaceutical Sciences, Mack Publishing Co. (A. R. Gennaro edit. 1985). The choice of pharmaceutical carrier, excipient or diluent can be selected with regard to the intended route of administration and standard pharmaceutical practice. The pharmaceutical compositions may comprise as—or in addition to—the carrier, excipient or diluent any suitable binder(s), lubricant(s), suspending agent(s), coating agent(s), solubilising agent(s).

Preservatives, stabilisers, dyes and even flavouring agents may be provided in the pharmaceutical composition. Examples of preservatives include sodium benzoate, sorbic acid and esters of p-hydroxybenzoic acid. Antioxidants and suspending agents may be also used.

There may be different composition/formulation requirements dependent on the different delivery systems. By way of example, the pharmaceutical composition of the present invention may be formulated to be delivered using a mini-pump or by a mucosal route, for example, as a nasal spray or aerosol for inhalation or ingestible solution, or parenterally in which the composition is formulated by an injectable form, for delivery, by, for example, an intravenous, intramuscular or subcutaneous route. Alternatively, the formulation may be designed to be delivered by both routes.

Where the agent is to be delivered mucosally through the gastrointestinal mucosa, it should be able to remain stable during transit though the gastrointestinal tract; for example, it should be resistant to proteolytic degradation, stable at acid pH and resistant to the detergent effects of bile.

Where appropriate, the pharmaceutical composition can be administered by inhalation, in the form of a suppository or pessary, topically in the form of a lotion, solution, cream, ointment or dusting powder, by use of a skin patch, orally in the form of tablets containing excipients such as starch or lactose, or in capsules or ovules either alone or in admixture with excipients, or in the form of elixirs, solutions or suspensions containing flavouring or colouring agents, or they can be injected parenterally, for example intravenously, intramuscularly or subcutaneously. For parenteral administration, the compositions may be best used in the form of a sterile aqueous solution which may contain other substances, for example enough salts or monosaccharides to make the solution isotonic with blood. For buccal or sublingual administration the compositions may be administered in the form of tablets or lozenges which can be formulated in a conventional manner.

For some embodiments, the agent of the present invention may also be used in combination with a cyclodextrin. Cyclodextrins are known to form inclusion and non-inclusion complexes with drug molecules. Formation of a drug-cyclodextrin complex may modify the solubility, dissolution rate, bioavailability and/or stability property of a drug molecule. Drug-cyclodextrin complexes are generally useful for most dosage forms and administration routes. As an alternative to direct complexation with the drug the cyclodextrin may be used as an auxiliary additive, e. g. as a carrier, diluent or solubiliser. Alpha-, beta and gamma-cyclodextrins are most commonly used and suitable examples are described in WO-A-91/11172, WO-A-94/02518 and WO-A-98/55148.

In one embodiment, the agent of the present invention are delivered systemically (such as orally, buccally, sublingually). In another embodiment the agent is delivered orally.

The compound of the invention may be milled using known milling procedures such as wet milling to obtain a particle size appropriate for tablet formation and for other formulation types. Finely divided (nanoparticulate) preparations of the compounds of the invention may be prepared by processes known in the art, for example see International Patent Application No. WO 02/00196 (SmithKline Beecham).

For oral administration, the pharmaceutical compositions may take the form of, for example, tablets or capsules prepared by conventional means with pharmaceutically acceptable excipients such as binding agents (e.g. pregelatinised maize starch, polyvinylpyrrolidone or hydroxypropyl methylcellulose); fillers (e.g. lactose, microcrystalline cellulose or calcium hydrogen phosphate); lubricants (e.g. magnesium stearate, talc or silica); disintegrants (e.g. potato starch or sodium starch glycollate); or wetting agents (e.g. sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g. sorbitol syrup, cellulose derivatives or hydrogenated edible fats); emulsifying agents (e.g. lecithin or acacia); non-aqueous vehicles (e.g. almond oil, oily esters, ethyl alcohol or fractionated vegetable oils); and preservatives (e.g. methyl or propyl-p-hydroxybenzoates or sorbic acid). The preparations may also contain buffer salts, flavouring, colouring and sweetening agents as appropriate.

Preparations for oral administration may be suitably formulated to give controlled release of the active compound.

For buccal administration the composition may take the form of tablets or formulated in conventional manner.

The compound of the invention may be formulated for parenteral administration by bolus injection or continuous infusion. Formulations for injection may be presented in unit dosage form e.g. in ampoules or in multi-dose containers, with an added preservative. The compositions may take such forms as suspensions, solutions or emulsions in oily or aqueous vehicles, and may contain formulatory agents such as suspending, stabilising and/or dispersing agents. Alternatively, the active ingredient may be in powder form for constitution with a suitable vehicle, e.g. sterile pyrogen-free water, before use.

The compound of the invention may be formulated for topical administration in the form of ointments, creams, gels, lotions, pessaries, aerosols or drops (e.g. eye, ear or nose drops). Ointments and creams may, for example, be formulated with an aqueous or oily base with the addition of suitable thickening and/or gelling agents. Ointments for administration to the eye may be manufactured in a sterile manner using sterilised components.

Lotions may be formulated with an aqueous or oily base and will in general also contain one or more emulsifying agents, stabilising agents, dispersing agents, suspending agents, thickening agents, or colouring agents. Drops may be formulated with an aqueous or non-aqueous base also comprising one or more dispersing agents, stabilising agents, solubilising agents or suspending agents. They may also contain a preservative.

The compound of the invention may also be formulated in rectal compositions such as suppositories or retention enemas, e.g. containing conventional suppository bases such as cocoa butter or other glycerides.

The compound of the invention may also be formulated as depot preparations. Such long acting formulations may be administered by implantation (for example subcutaneously or intramuscularly) or by intramuscular injection. Thus, for example, the compounds of the invention may be formulated with suitable polymeric or hydrophobic materials (for example as an emulsion in an acceptable oil) or ion exchange resins, or as sparingly soluble derivatives, for example, as a sparingly soluble salt.

For intranasal administration, the compound of the invention may be formulated as solutions for administration via a suitable metered or unitary dose device or alternatively as a powder mix with a suitable carrier for administration using a suitable delivery device.

A proposed dose of the compound of the invention is 1 to about 1000 mg per day. It will be appreciated that it may be necessary to make routine variations to the dosage, depending on the age and condition of the patient and the precise dosage will be ultimately at the discretion of the attendant physician or veterinarian. The dosage will also depend on the route of administration and the particular compound selected.

Thus for parenteral administration a daily dose will typically be in the range of 1 to about 100 mg, preferably 1 to 80 mg per day. For oral administration a daily dose will typically be within the range 1 to 300 mg e.g. 1 to 100 mg.

The CRF antagonists compounds which can be prepared by the process object of the present invention may be in the form of a pharmaceutically acceptable salt. For a review on suitable salts see Berge et al, J. Pharm. Sci., 1977, 66, 1-19.

Typically, a pharmaceutical acceptable salt may be readily prepared by using a desired acid or base as appropriate. The salt may precipitate from solution and be collected by filtration or may be recovered by evaporation of the solvent.

Suitable addition salts are formed from acids which form non-toxic salts and examples are hydrochloride, hydrobromide, hydroiodide, sulphate, bisulphate, nitrate, phosphate, hydrogen phosphate, acetate, maleate, malate, fumarate, lactate, tartrate, citrate, formate, gluconate, succinate, piruvate, oxalate, oxaloacetate, trifluoroacetate, saccharate, benzoate, methansulphonate, ethanesulphonate, benzenesulphonate, p-toluensulphonate, methanesulphonic, ethanesulphonic, p-toluenesulphonic, and isethionate.

Pharmaceutically acceptable base salts include ammonium salts, alkali metal salts such as those of sodium and potassium, alkaline earth metal salts such as those of calcium and magnesium and salts with organic bases, including salts of primary, secondary and tertiary amines, such as isopropylamine, diethylamine, ethanolamine, trimethylamine, dicyclohexyl amine and N-methyl-D-glucamine.

Those skilled in the art of organic chemistry will appreciate that many organic compounds can form complexes with solvents in which they are reacted or from which they are precipitated or crystallized. These complexes are known as “solvates”. For example, a complex with water is known as a “hydrate”. Solvates of the compound of the invention are within the scope of the invention.

The compounds of formula (I) may readily be isolated in association with solvent molecules by crystallisation or evaporation of an appropriate solvent to give the corresponding solvates.

When a specific enantiomer of a compound of general formula (I) is required, this may be obtained for example by resolution of a corresponding enantiomeric mixture of a compound of formula (I) using conventional methods. Thus the required enantiomer may be obtained from the racemic compound of formula (I) by use of chiral HPLC procedure.

### EXAMPLES

In the Intermediates and Examples unless otherwise stated:

All temperatures refers to ° C. Infrared spectra were measured on a FT-IR instrument. Compounds were analysed by direct infusion of the sample dissolved in acetonitrile into a mass spectra operated in positive electro spray (ES⁺) ionisation mode. Proton Magnetic Resonance (¹H-NMR) spectra were recorded at 400 MHz, chemical shifts are reported in ppm downfield (d) from Me₄Si, used as internal standard, and are assigned as singlets (s), broad singlets (bs), doublets (d), doublets of doublets (dd), triplets (t), quartets (q) or multiplets (m). A strategy comprising of NOE (Nuclear Overhauser Effect) correlation and/or 1H,15N long range scalar correlations measurements has been implemented in order to allow elucidation of possible regio-isomers structure of compounds of the present invention. Proposed structures were verified by measurement of the vicinity in the space of key hydrogens, thus 1 D Nuclear Overhauser difference spectra were used to measure 1H,1H-dipole-dipole correlations.

In cases where NOE measurements were not conclusive, 1H,15N long range scalar correlations were measured via 1H,15N-HMBC experiments. A delay corresponding to an average long range scalar coupling 2,3 J(1H,15N) of 6 Hz was set for optimal result. Column chromathography was carried out over silica gel (Merck AG Darmstaadt, Germany). The following abbreviations are used in the text: EtOAc=ethyl acetate, cHex=cyclohexane, CH₂Cl₂=DCM, dichloromethane, Et₂O=dietyl ether, DMF=N,N′-dimethylformamide, DIPEA=N,N-diisopropylethylamine, DME=ethylene glycol dimethyl ether, MeOH=methanol, Et₃N=triethylamine, TFA=trifluoroacetic acid, THF=tetrahydrofuran, KOtBu=potassium tert-butoxide, NMP=N-methyl-2-pyrrolidinone, MTBE=methyl-tert-butyl ether, IPA=isopropanol, DABCO=Diazabicyclo[2.2.2]octane, DBU=1,8-Diazabicyclo[5.4.0]undecen-7-ene, BINAP=2,2′-Bis(diphenylphosphino)-1,1′-binaphthyl, Acac=2,4-Pentanedione, MEK=methyl ethyl ketone.

The method HPLC used for the purity determination is the following:

| | |
|---|---|
| Column | Phenomenex Luna 3μ C18(2) - 50 × 2.0 mm |
| Wavelength | 220 nm |
| Flow | 1 mL |
| Injection volume | 5 uL (2 uL) |
| Temperature | 40° C. |
| Run Time | 10 min |
| Sample conc. | ca. 0.5 mg/mL (ca. 1 mg/mL) |
| Mobile Phase | Solution A: 0.05% TFA in water |
| | Solution B: 0.05% TFA in ACN |
| Gradient | FAST gradient: |
| | 0.00-8.00 min: . . . from 100% A down to 5% |
| | 8.01-8.10 min: . . . from 5% A up to 100% |
| | 8.11-10.00 min: . . . 100% of A |

### Example 1

### Preparation of Intermediate (III)

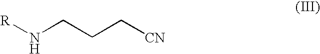

A solution of tertiary amines (e.g. TEA, DIPEA; 1 eq) and RNH₂ (1 eq.) in polar aprotic solvent (e.g. DMF, NMP) was heated to 100-150° C. 4-X-butyrronitrile, where X═Cl or Br; 1 eq) was added dropwise under N₂. The reaction mixture was heated for 2-6 hr. The mixture was cooled down to R.T. and diluted with ether (e.g. MTBE, Et₂O). Water was added and the phases were separated. The organic layer was further washed with water and evaporated to low volume. New ether was added and the mixture again evaporated to low volume. The mixture was treated with cyclic alcanes (e.g cyclohexane) over 20 minutes and the resulting suspension aged at room temperature for 1-5 hr. The suspension was filtered and the cake washed with a mixture ether/alcane mixture. The title compound was collected as a solid.

4-{[2-methyl-4-(methyloxy)phenyl]amino}butanenitrile

Yield: 65-70% th

4-{[2-methyl-6-(methyloxy)-3-pyridinyl]amino}butanenitrile

Yield: 80%

All the analytical data are set forth in the following Table 1-1

TABLE 1-1

| Cpd. | | |
|---|---|---|
| No. | R | Analytical Data |
| 1-1 | 2-methyl-4-methoxy- | NMR (¹H, CDCl₃): NMR (¹H, |
| | phenyl | DMSO-d₆): δ 6.65 (d, 1H), |
| | | 6.63 (dd, 1H), 6.47 (d, 1H), 4.49 (bt, |
| | | 1H), 3.64 (s, 3H), 3.10 (q, 2H), |
| | | 2.59 (t, 2H), 2.09 (s, 3H), |
| | | 1.86 (m, 2H). |
| | | M/S (m/z): 205 [MH]⁺ |
| | | HPLC % a/a > 99 |
| 1-2 | 2-methyl-4-methoxy- | NMR (¹H, DMSO-d₆): δ 6.94 (d, |
| | 3-piridynyl | 1H), 6.53 (d, 1H), 3.85 (s, 3H), |
| | | 3.28 (t, 2H), 3.14 (bs, 1H), |
| | | 2.50 (t, 2H), 2.33 (s, 3H), 1.97 (m, |
| | | 2H) |
| | | M/S (m/z): 206 [MH]⁺ |
| | | HPLC % a/a 81% |

### Example 2

### Preparation of Intermediates (IV)

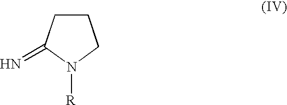

To a suspension of intermediate (III) in alcoholic solvents (e.g IPA), aromatic solvents (e.g. Toluene) or etheral solvents (e.g THF), an organic acid (e.g. p-toluenesulfonic acid; methanesulfonic acid) or a mineral acid (e.g. HCl 5-6N in IPA) (1.5 eq) was added at R.T. under N₂. The mixture was heated to reflux for 4-8 hr, allowed to cool down to R.T. and evaporated to low volume. Water was added, the clear solution evaporated again to low volume and treated with NaOH aqueous solution. The mixture was extracted with organic solvent (DCM, ethyl acetate) and the organic layer further washed with NaCl aqueous solution. The organic layer was evaporated down to dryness. The crude product was used as such in the next step.

1-[2-methyl-4-(methyloxy)phenyl]-2-pyrrolidinimine

4-{[2-Methyl-4-(methyloxy)phenyl]amino}butanenitrile (0.78 Kg; 1 eq) was treated with HCl 10% in water (2.34 L) and the solution heated to 85° C. After 4 hour the mixture was cooled down to 20° C., diluted with NaOH 10% and extracted with DCM. The aqueous layer was further extracted with DCM. The combined organic layers were washed with NaCl 15%. The collected organic phase was diluted with THF, distilled to about 1 L volume (50° C. jacket, 250 mbar). THF was added and the mixture distilled again to about 1 L. Fresh THF was added one more time and the mixture again distilled down to about 4 L. The product is used as such in the next step.

Yield: 95-99% th

1-[2-methyl-6-(methyloxy)-3-pyridinyl]-2-pyrrolidinimine

Yield: 78% th

All the analytical data are set forth in the following Table 2-1

TABLE 2-1

| Cpd. | | |
|---|---|---|
| No. | R | Analytical Data |
| 2-1 | 2-methyl-4-methoxy- | NMR (¹H, DMSO-d₆): δ 7.09 (d, |
| | phenyl | 1H), 6.87 (d, 1H), 6.80 (dd, 1H), |
| | | 5.8-5.4 (b, 1H), 3.75 (s, 1H), |
| | | 3.54 (t, 2H), 2.51 (t, 2H), |
| | | 2.11 (s, 3H), 2.01 (m, 2H). |
| | | M/S (m/z): 205 [MH]⁺ |
| | | HPLC % a/a > 98 |
| 2-2 | 2-methyl-4-methoxy- | NMR (¹H, DMSO-d₆): δ 7.53 (d, |
| | 3-piridynyl | 1H), 6.68 (d, 1H), 6.1-5.8 (b, |
| | | 1H), 3.84 (s, 3H), 3.55 (t, 2H), |
| | | 2.50 (m, 2H), 2.26 (s, 3H), |
| | | 2.02 (m, 2H) |
| | | M/S (m/z): 205 [MH]⁺ |
| | | HPLC % a/a 86% |
| 2-3 | 2-methyl-4- | NMR (¹H, CDCl₃): δ 7.84 (d, |
| | trifluormethoxy- | 1H), 7.30 (d, 1H), 7.12 (s, 1H), |
| | phenyl | 7.09 (d, 1H), 6.98 (d, 1H), |
| | | 6.63 (s, 1H), 4.68 (s, 1H), 4.10 (t, |
| | | 2H), 3.91 (t, 2H), 3.63 (t, 2H), |
| | | 3.50 (t, 2H), 2.36 (s, 3H), |
| | | 2.29 (s, 3H). |
| | | MS (m/z): 459 [MH]⁺ |

### Example 3

### Preparation of Compounds (IVB)

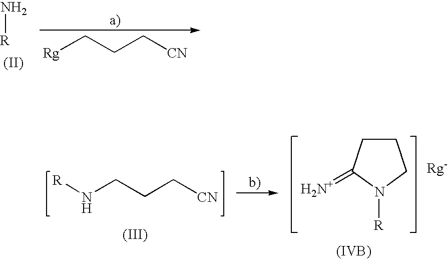

1-[2-methyl-4-(trifluoromethyloxy)phenyl]-2-pyrrolidinimine hydrobromide

2-Methyl-4-trifluoromethyloxyaniline (30 g) was dissolved in NMP (90 ml). The resulting solution was heated up to 100° C. Neat bromobutyrronitrile (1.1 eq; 17.2 mL) was then added and the resulting solution was heated at 115-118° C. for 2-4 hours.

The reaction was then allowed to cool to 45° C. in 30 min. A seed of the desired compound (0.03 g) was added. MTBE (270 ml) was added at 45° C. in 30-40 min. The resulting suspension was cooled to 20° C. in 20 min, stirred for 2 hrs and then was filtered. The cake was washed with a mixture of 3:1 MTBE/NMP (3×60 mL) and the solid dried overnight at 70° C. for 6 hrs.

Yield: 88% th from 2-Methyl-4-trifluoromethyloxyaniline

NMR (1H, DMSO-d6): 9.83 (s, 1H), 8.62 (s, 1H), 7.58 (d, 1H), 7.48 (d, 1H), 7.41 (dd, 1H), 3.92 (t, 2H), 3.08 (m, 2H), 2.24 (m, 2H), 2.24 (s, 3H).

HPLC % a/a 99%

### Example 4

### Preparation of Intermediates (V)

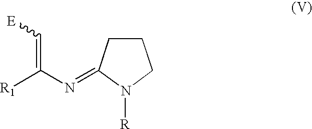

To a solution of intermediate (IV) in etheral solvent (e.g. THF), polar aprotic solvents (e.g. acetonitrile), or alcoholic solvents (e.g. IPA); ethyl-2-butynoate (1.0-1.5 eq) was added at R.T. under N₂. The mixture was heated to reflux and aged for 2-20 hr before allowing cooling down to R.T. The reaction mixture was evaporated to dryness. The crude oil was used as such in the next step.

Ethyl-3-({(2E)-1-[2-methyl-4-(methyloxy)phenyl]-2-pyrrolidinylidene}amino)-2-butenoate

The solution containing 1-[2-methyl-4-(methyloxy)phenyl]-2-pyrrolidinimine, as previously prepared, was treated with ethyl-2-butynoate (1.1 eq, 0.49 L). The mixture was heated to reflux for 12-14 hours. The mixture was allowed to cool to room temperature. The product is used as such in the next step.

Yield 80-90% th

Ethyl-3-({(2E)-1-[2-methyl-6-(methyloxy)-3-pyridinyl]-2-pyrrolidinylidene}amino)-2-butenoate

Yield 89% th

Ethyl-3-({(2E)-1-[2-methyl-4-(trifluoromethyloxy)phenyl]-2-pyrrolidinylidene}amino-2-butenoate

1-[2-methyl4-(trifluoromethyloxy)phenyl]-2-pyrrolidinimine hydrobromide (1.4 kg) was treated with 10% NaOH acq. solution (4.2 L) and extracted with DCM (4.2 L). The aqueous layer was further extracted with DCM (2.8 L). The combined organic layers were washed with aqueous sodium chloride w/v 15% (5.6 L). The collected organic phase was diluted with toluene (7 L), distilled to 2.8 L, diluted with toluene (14 L) and distilled to 2.8 L. The solution was treated with ethyl-2-butynoate (1.1 eq, 0.53 L). The mixture was heated to reflux for about 9 hours. The mixture was allowed to cool to room temperature. The product is used as such in the next step.

All the analytical data are set forth in the following Table 3-1

TABLE 3-1

| Cpd. | | |
|---|---|---|
| No. | R | Analytical Data |
| 3-1 | 2-methyl-4-methoxy- | M/S (m/z): 317 [MH]⁺ |
| | phenyl | HPLC % a/a > 90 |
| 3-2 | 2-methyl-4-methoxy- | M/S (m/z): 318 [MH]⁺ |
| | 3-piridynyl | HPLC % a/a 79% |

### Example 5

### Preparation of Intermediates (VI)

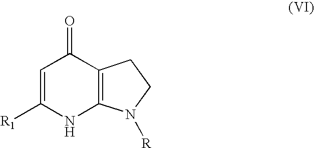

To a solution of intermediate (V) in etheral solvents (e.g THF), alcoholic solvents (e.g. IPA), polar aprotics solvents (e.g. acetonitrile, DMF) was added at R.T. under N₂, a base (e.g. t-BuOK, LiHMDS, DABCO, DBU, NaH). The reaction mixture was heated to reflux and stirred for 2-14 hr. The solution was allowed to cool down to R.T., evaporated to low volume and diluted with chlorinated solvent (e.g. DCM). The organic layer was washed with sat.aq. NH₄Cl; followed by NaCl aqueous solution. The organic layer was evaporated to dryness and the crude product was used as such in the next step.

6-methyl-1-[2-methyl-4-(methyloxy)phenyl]-1,2,3,7-tetrahydro-4H-pyrrolo[2,3-b]pyridin-4-one

The solution from the previous step containing ethyl-3-({(2E)-1-[2-methyl-4-(methyloxy)phenyl]-2-pyrrolidinylidene}amino)-2-butenoate was treated with t-BuOK 1 M in THF (7.8 L; prepared by dissolution of solid tBuOK-2eq-in THF). The t-BuOK solution was added the first 20% in 30 minutes and the remaining part in 40-50 minutes. The mixture was refluxed for 6 hours. Then it was cooled to 20° C., concentrated (50° C. jacket, 300-250 mbar), diluted with NH₄Cl sat. sol. and extracted with DCM. The aqueous layer was back extracted with DCM. The combined organic phases were washed with NaCl 15%. The organic layer was distilled down to about 1 L, diluted with MEK and evaporated down to about 4 L. Fresh MEK was added and the mixture was concentrated down to about 4 L. The product is used as such in the next step.

Yield 75-85% th

6-methyl-1-[2-methyl-6-(methyloxy)-3-pyridinyl]-1,2,3,7-tetrahydro-4H-pyrrolo[2,3-b]pyridin-4-one

Yield: 15-20% th

6-methyl-1-[2-methyl-4-(trifluoromethyloxy)phenyl]-1,2,3,7-tetrahydro-4H-pyrrolo[2,3-b]pyridin-4-one

The solution from the previous step containing ethyl-3-({(2E)-1-[2-methyl-4-(trifluoromethyloxy)phenyl]-2-pyrrolidinylidene}amino)-2-butenoate was treated with t-BuOK 1 M in THF (8.26 L; prepared by dissolution of solid tBuOK-2eq-in THF). The t-BuOK solution was added in 30 minutes. The mixture was refluxed for 3 hours. Then it was cooled to 20° C., concentrated to 4.2 L (50° C. jacket, 300-250mbar), diluted with NH₄CI sat. sol. (7 L) and extracted with DCM (11.2 L).The aqueous layer was back extracted with DCM (4.2 L) The combined organic phases were washed with NaCl 15% (2.8 L). The organic layer was distilled down to 2.8 L (50° C. jacket, 300 mbar), diluted with THF (11.2 L) and evaporated down to 2.8 L. Fresh THF (7 L) was added. The solution was treated with CH₃SO₃H (0.28 L) in a dropwise fashion over 1 hr. Precipitation occurred during the addition of the acid. The suspension was aged for 4-6 hours, then filtered and the cake washed with THF (5.6 L). The collected solid was placed in the oven at 70° C., under reduced pressure for at least 5-6 hours.

Overall yield: 50-65%

All the analytical data are set forth in the following Table 4-1

TABLE 4-1

| Cpd. | | |
|---|---|---|
| No. | R | Analytical Data |
| 4-1 | 2-methyl-4-methoxy- | NMR (¹H, DMSO-d₆): δ 9.8 (b, |
| | phenyl | 1H), 7.08 (d, 1H), 6.80 (d, 1H), |
| | | 6.75 (dd, 1H), 5.92 (s, 1H), |
| | | 3.72 (s, 3H), 3.68 (t, 2H), 2.89 (t, |
| | | 2H), 2.12 (s, 3H), 2.02 (s, 3H). |
| | | M/S (m/z): 271 [MH]⁺ |
| | | HPLC % a/a 75-80 |
| 4-2 | 2-methyl-4-methoxy- | NMR (¹H, DMSO-d₆): δ |
| | 3-piridynyl | 9.93 (bs, 1H), 7.55 (d, 1H), 6.67 (d, |
| | | 1H), 5.99 (s, 1H), 3.84 (s, 3H), |
| | | 3.73 (t, 2H), 2.94 (t, 2H), |
| | | 2.28-2.07 (2s, 6H). |
| | | M/S (m/z): 272 [MH]⁺ |
| | | HPLC % a/a 35-40 |

### Example 6

### Preparation of Intermediates (VII)

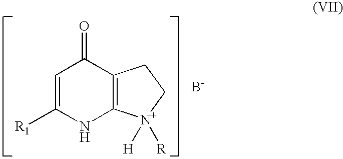

Intermediate (VI) was dissolved in etheral solvents (e.g THF), ketonic solvents (e.g acetone, 2-butanone), treated with sulphonic acid (e.g p-toluensulphonic; methanesulphonic, triflic anhydride) and seeded with intermediate (VII). After 2-10 hr the suspension was filtered and the cake washed with further solvent. The collected solid was placed in the oven at 40° C. under reduced pressure for 10-24 hr.

6-methyl-1-[2-methyl-4-(methyloxy)phenyl]-1,2,3,7-tetrahydro-4H-pyrrolo[2,3-b]pyridin-4-one methanesulfonate

The solution containing 6-methyl-1-[2-methyl-4-(methyloxy)phenyl]-1,2,3,7-tetrahydro-4H-pyrrolo[2,3-b]pyridin-4-one as previously prepared, was treated with CH₃SO₃H (0.187 L) in a dropwise fashion over 20-25 minutes (temperature rose from 20° C. to 30° C. internally) and seeded with the title compound. Precipitation occurred soon after seeding. The suspension was aged for 6 hours, then filtered and the cake washed with 2-butanone. The collected solid was placed in the oven at 40° C., under reduced pressure for 10-12 hours.

Yield: 90-95% th

6-methyl-1-[2-methyl-4-(methyloxy)phenyl]-1,2,3,7-tetrahydro-4H-pyrrolo[2,3-b]pyridin-4-one 4-methylbenzenesulfonate

Yield: 54% th

6-methyl-1-[2-methyl-4-(trifluoromethyloxy)phenyl]-1,2,3,7-tetrahydro-4H-pyrrolo[2,3-b]pyridin-4-one trifluoromethanesulfonate

A saturated aqueous solution of NaHCO₃ (6 L) was added at room temperature to a suspension of 6-methyl-1-[2-methyl-4-(trifluoromethyloxy)phenyl]-1,2,3,7-tetrahydro-4H-pyrrolo[2,3-b]pyridin-4-one (1 Kg) in dichloromethane (10 L). The resulting mixture was stirred for 20 min at room temperature. The separated organic phase was washed with a 15% (w/v) aqueous solution of NaCl (3 L), then was diluted with CH₂Cl₂ (10 L). The resulting solution was distilled down to 10 L. Fresh CH₂Cl₂ (5 L) was added and the solution was concentrated to 10 L. Fresh CH₂Cl₂ (5 L) was added and the solution was concentrated again to 10 L. The solution as such is used in the next step.

All the analytical data are set forth in the following Table 5-1

TABLE 5-1

| Cpd. | | |
|---|---|---|
| No. | R | Analytical Data |
| 5-1 | 2-methyl-4-methoxy- | NMR (¹H, DMSO-d₆): δ |
| | phenyl | 12.36-12.70 (2bs, 2H), 7.33 (d, 1H), |
| | | 7.00 (d, 1H), 6.92 (dd, 1H), |
| | | 6.26 (s, 1H), 4.02 (bm, 2H), |
| | | 3.81 (s, 3H), 3.11 (m, 2H), |
| | | 2.34 (s, 3H), 2.25-2.20 (2s, 6H). |
| | | HPLC a/a > 98 |
| 5-2 | 2-methy-4-methoxy- | NMR (¹H, DMSO-d₆): δ |
| | 3-piridynyl | 12.34-12.00 (2bs, 2H), 7.50 (d, 2H), |
| | | 7.32 (d, 1H), 7.13 (d, 2H), |
| | | 7.00 (d, 1H), 6.92 (dd, 1H), 6.26 (s, |
| | | 1H), 4.02 (m, 2H), 3.80 (s, 3H), |
| | | 3.11 (m, 2H), 2.30 (s, 3H), |
| | | 2.24-2.19 (2s, 6H). |
| | | HPLC a/a > 98 |

### Example 7

### Preparation of Intermediates (VIII)

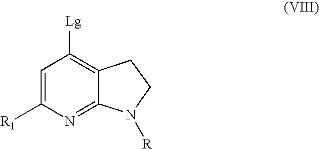

To a suspension of intermediate (VII) in chlorinated solvents (e.g. DCM), an inorganic base in aqueous solution was added. After phase separation the organic one was washed with NaCl aqueous solution and dried. An amine (e.g. pyridine, TEA) was added at R.T. under N₂ at the organic solution. The mixture was cooled down to low temperature (below −10° C.) and triflic anhydride or methanesulfonic anhydride or methanesulfonyl chloride was added in a dropwise fashion. The reaction mixture was allowed to warm up to 5° C. over 30 minutes and treated with sat.aq NaHCO₃. Phases were separated and the organic one was further washed with water and concentrated to oil. The oil was dissolved in alcoholic solvent (IPA) and seeded with intermediate (VIII). The suspension was stirred for 1-4 hr, then water was added over 30 minutes and the mixture aged for additional 1-5 hr. The suspension was filtered, the cake washed with an alcohol/water mixture 1/1, collected and dried in the oven at 35-40° C. under high vacuum for 12-14 hours. The title compound was obtained as a solid.

6-methyl-1-[2-methyl-4-(methyloxy)phenyl]-2,3-dihydro-1H-pyrrolo[2,3-b]pyridin-4-yl trifluoromethanesulfonate

6-Methyl-1-[2-methyl-4-(methyloxy)phenyl]-1,2,3,7-tetrahydro-4H-pyrrolo[2,3-b]pyridin-4-one methanesulfonate as previously prepared (0.4 Kg; 1 eq) was suspended in DCM (4 L) and treated with NaHCO₃ sat. sol. (2.4 L). Phases were allowed to separate and the organic one washed with NaCl 15%. The organic layer was diluted with DCM and the solution distilled down to 4 L. Fresh DCM added again and the mixture distilled down to 4 residual litres. The solution was treated with Pyridine (0.097 L, 1.1 eq) and cooled down to −15° C. Triflic anhydride (0.193 L, 1.05 eq) was added over 60 min keeping the temperature below −10° C. The mixture was allowed to warm up to 5° C. over 20 min and quenched with NaHCO₃ sat. over 20 minutes keeping temperature at 5° C. The biphasic mixture was allowed to warm up to R.T. while stirring for additional 20 minutes to complete CO₂ evolution; then allowed to separate. The organic one further washed with water, distilled down to 1.6 L (50° C. jacket, 250 mbar) and diluted with IPA. The solution was distilled down to about 2 L (50° C. jacket, 100-150 mbar), diluted with fresh IPA and again distilled down to about 2 L (50° C. jacket, 100-150 mbar). The solution was brought to room temperature and seeded with the title compound. The slurry was aged for 60 min. Water was added over 30 min and the resulting suspension aged for 90 min before being filtered. The cake was washed with IPA-water 1:1, collected and placed in the oven at 35° C., under reduced pressure overnight.

Yield: 80-85% th

6-methyl-1-[2-methyl-4-(methyloxy)phenyl]-2,3-dihydro-1H-pyrrolo[2,3-b]pyridin-4-yl methanesulfonate

Yield: 82% th

6-methyl-1-[2-methyl-4-(trifluoromethyloxy)phenyl]-2,3-dihydro-1H-pyrrolo[2,3-b]pyridin-4-yl trifluoromethanesulfonate

Pyridine (1.1 equiv, 0.21 L) was added to the solution containing 6-methyl-1-[2-methyl-4-(trifluoromethyloxy)phenyl]-1,2,3,7-tetrahydro-4H-pyrrolo[2,3-b]pyridin-4-one trifluoromethanesulfonate and the resulting mixture was cooled down to −15 ° C. Neat trifluoromethanesulfonic anhydride (1.05 equiv, 0.41 L) was then added dropwise maintaining, the temperature ranging below −10 ° C., then the solution was heated up to 5° C. in 40 min. A saturated aqueous solution of NaHCO₃ (5 L) was then added dropwise in 30 min, keeping the temperature below 5 OC. The solution was finally heated up to 20° C. in 30 min. The separated organic layer was then washed with water (5 L) and concentrated to 4 L. Fresh IPA (8 L) was then added and the resulting solution was distilled down to 8 L. Fresh IPA (8 L) was added and the solution was distilled down to 8 L. The solution was cooled down to room temperature. A yellow solid precipitated at room temperature. The resulting suspension was stirred for 0.5 h at room temperature, then water (8 L) was added and the suspension was stirred overnight, filtered and the solid was washed with a mixture of IPA/water 1:1 (2×2 L) and dried overnight at 40° C. under high vacuum.

Overall yield: 80-95%

All the analytical data are set forth in the following Table 6-1

TABLE 6-1

| Cpd. | | |
|---|---|---|
| No. | R | Analytical Data |
| 6-1 | 2-methyl-4-methoxy- | NMR (¹H, DMSO-d₆): δ 7.17 (d, |
| | phenyl | 1H), 6.85 (d, 1H), 6.77 (dd, 1H), |
| | | 6.40 (s, 1H), 3.89 (t, 2H), |
| | | 3.73 (s, 3H), 3.16 (t, 2H), |
| | | 2.17-2.11 (2s, 6H) |
| | | M/S (m/z): 403 [MH]⁺ |
| | | HPLC % a/a > 99 |
| 6-2 | 2-methyl-4-methoxy- | NMR (¹H, DMSO-d₆): δ 7.22 (d, |
| | 3-piridynyl | 1H), 6.89 (d, 1H), 6.82 (dd, 1H), |
| | | 6.49 (s, 1H), 3.91 (t, 2H), |
| | | 3.76 (s, 3H), 3.52 (s, 3H), 3.18 (t, |
| | | 2H), 2.20 (s, 3H), 2.15 (s, 3H) |
| | | M/S (m/z): 349 [MH]⁺ |
| | | HPLC % a/a > 99 |

### Example 8

### General Preparation of Compounds of Formula (I)

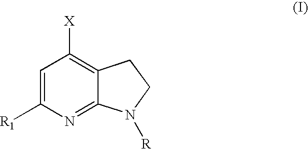

To a solution of intermediate (VIII) in polar aprotic solvents (e.g. DMF, NMP, acetonitrile), alcoholic solvents (e.g. IPA) or apolar solvents (e.g. toluene) was added an organic acid (e.g. methansulfonic acid, acetic acid, p-toluenesulfonic acid, trifluoroacetic acid, fumaric acid) followed by a halide salt (e.g. LiX, NaX, KX; X═Cl, Br, I) and the resulting mixture was heated at 50-120° C. for 2-24 hr.

The mixture was allowed to cool down to R.T. and diluted with ethereal or estereal solvents (e.g. MTBE, AcOEt) and washed with NaOH 1 N; the organic phase was washed twice with water then dried over Na₂SO₄. The removal of solvents under reduced pressure gave the intermediate (VIII) that was used as such in the next step.

3-Chloro-6-methyl-1-[2-methyl-4-(methyloxy)phenyl]-2,3-dihydro-1H-pyrrolo[2,3-b]pyridine

Yield: 85-95% th

3-Bromo-6-methyl-1-[2-methyl-4-(methyloxy)phenyl]-2,3-dihydro-1H-pyrrolo[2,3-b]pyridine

To a solution of 6-methyl-1-[2-methyl-4-(methyloxy)phenyl]-2,3-dihydro-1H-pyrrolo[2,3-b]pyridin-4-yl trifluoromethanesulfonate as previously prepared (1.2 Kg, 1.0 eq) in DMF (4.2 L) under a N₂ atmosphere, CH₃SO₃H (232.25 ml) was added followed by sodium bromide (460.33 g). The resulting mixture was heated at 85° C. for 2.5 h.

The mixture was diluted with MTBE and washed with NaOH 1 N; the aqueous phase was extracted again with MTBE and the combined organic phases washed twice with water. The organic layer was distilled down to 3.0 L (50° C. jacket, 500 mbar), diluted with fresh DMF and again distilled down to 3.0 L (50° C. jacket, 100-150mbar). The DMF solution was used as such in the next step.

Yield 85-95% th

3-Iodo-6-methyl-1-[2-methyl-4-(methyloxy)phenyl]-2,3-dihydro-1H-pyrrolo[2,3-b]pyridine

To a solution of 6-methyl-1-[2-methyl-4-(methyloxy)phenyl]-2,3-dihydro-1H-pyrrolo[2,3-b]pyridin-4-yl trifluoromethanesulfonate (300 g, 1.0 eq) in NMP (1.05 L) under a N₂ atmosphere, CH₃SO₃H (58.06 mL) was added followed by potassium iodide (185.7 g).

The resulting mixture was heated at 85° C. for 7 h.

The mixture was diluted with AcOEt and washed with NaOH 1 N; the organic phases washed twice with water. The organic layer was distilled down to about 1 L (50° C. jacket, 500 mbar), diluted with fresh NMP and again distilled down to about 1 L (50° C. jacket, 100-150mbar). The NMP solution was used as such in the next step. The HPLC purity was greater then 92% a/a.

Yield: 85-95% th

4-Iodo-6-methyl-1-[2-methyl-6-(methyloxy)-3-pyridinyl]-2,3-dihydro-1H-pyrrolo[2,3-b]pyridine

The title compound may be prepared according to the procedure described above.

3-Iodo-6-methyl-1-[2-methyl-4-(trifluoromethyloxy)phenyl]-2,3-dihydro-1H-pyrrolo[2,3-b]pyridine

To a solution of 6-methyl-1-[2-methyl-4-(trifluoromethyloxy)phenyl]-2,3-dihydro-1H-pyrrolo[2,3-b]pyridin-4-yl trifluoromethanesulfonate (0.4 kg) in NMP (1.6 L) under a N₂ atmosphere, CH₃SO₃H (0.068 L, 1.2 equiv) was added followed by potassium iodide (2.0 eq, 0.291 kg). The resulting mixture was heated to 90° C. for 2 hrs.

The mixture was cooled down to 25° C., diluted with AcOEt (4 L) and washed with NaOH 1 N (2 L) to reach pH=8-9, then the organic layer was washed 2 times with water (1.6 L). The organic layer was distilled down to 1.2 L, further ethyl acetate was added (2 L) diluted and the mixture was distilled down to 1.2 L. NMP (0.8 L) was added and again distilled down to 1.2 L. The NMP solution was used s such in the next step.

The HPLC purity was greater then 95% a/a.

All the analytical data are set forth in the following Table 7-1

TABLE 7-1

| Cpd. | | | |
|---|---|---|---|
| No. | X | R | Analytical Data |
| 7-1 | Cl | 2-methyl-4-methoxy- | NMR (¹H, DMSO-d₆): δ 7.17 (d, |
| | | phenyl | 1H), 6.87 (d, 1H), 6.80 (dd, 1H), |
| | | | 6.43 (s, 1H), 3.86 (t, 2H), |
| | | | 3.76 (s, 3H), 3.11 (t, 2H), 2.14 (2s, |
| | | | 6H) |
| | | | M/S (m/z): 289/291 [MH]⁺ |
| | | | HPLC % a/a > 92% |
| 7-2 | Br | 2-methyl-4-methoxy- | NMR (¹H, DMSO-d₆): δ 7.17 (d, |
| | | phenyl | 1H), 6.87 (d, 1H), 6.80 (dd, 1H), |
| | | | 6.56 (s, 1H), 3.86 (t, 2H), |
| | | | 3.76 (s, 3H), 3.06 (t, 2H), |
| | | | 2.15-2.14 (2s, 6H) |
| | | | M/S (m/z): 333/335 [MH]⁺ |
| | | | HPLC % a/a > 90% |
| 7-3 | I | 2-methyl-4-methoxy- | NMR (¹H, DMSO-d₆): δ 7.17 (d, |
| | | phenyl | 1H), 6.87 (d, 1H), 6.80 (dd, 1H), |
| | | | 6.56 (s, 1H), 3.86 (t, 2H), |
| | | | 3.76 (s, 3H), 3.06 (t, 2H), |
| | | | 2.15-2.14 (2s, 6H) |
| | | | M/S (m/z): 381 [MH]⁺ |
| | | | HPLC % a/a > 90% |

### Example 9

### General Preparation of Compounds of Formula (IA)

A solution of a copper catalyst (e.g. CuI, CuBr, Cu₂Br, Cu(AcO)₂, Cu₂O) and a ligand (e.g. cis- or trans-N,N′-dimethyl-1,2-cyclohexanediamine, a mixture of cis- and trans-N,N′-dimethyl-1,2-cyclohexanediamine, cis- or trans-1,2-cyclohexanediamine, a mixture of cis- and trans-1,2-cyclohexanediamine, N,N′-dimethyl-1,2-diaminoethane, NN,N′N′-tetramethyl-1,2-diaminoethane, ethanolamine, 1,10-phenantroline, PPh₃, BINAP, Acac) was prepared in a suitable solvent (e.g. DMF, NMP, DMSO, acetonitrile, dioxane, toluene).

Then an inorganic or organic base (e.g. potassium carbonate, cesium carbonate, potassium phosphate, ter-BuOK, DBU, TEA, DIPEA) was added followed by the Z-W-reactive derivative and the intermediate (VIII). The resulting mixture was heated at 80°-150° C. for 4-48 hr.

The mixture was cooled at 60° C. and water was added dropwise. The suspension was stirred at room temperature for 1 hr, then the white precipitate was filtered and washed upon the filter once with a 1/2 mixture of DMF/water, then twice with water. The solid was dried at 80° C. for 24hr to obtain the title compound as crude.

The crude was dissolved at room temperature in a suitable mixture, such as DCM/MeOH 9/1. The solution was filtered through a carbon pad washing upon the filter with a DCM/MeOH mixture 9/1. The mixture underwent a solvent exchange into a suitable solvent such as alcohols (e.g. Methanol) or aromatic ether (e. g. Anisole). The resulting suspension was aged for 2 hr, filtered and washed upon the filter with MeOH. The collected solid was dried at 80° C. for 24 hr to obtain the title compound.

Preparation of 6-Methyl-1-[2-methyl-4-(methyloxy)phenyl]-4-[3-(1,3-thiazol-2-yl)-1-H-pyrazol-1-yl]-2,3-dihydro-1H-pyrrolo[2,3-b]pyridine

2-(1H-pyrazol-3-yl)-1,3-thiazole

In a 2 liters flask formamide dimethylacetal (1 eq, 1.027 L) was treated, in a dropwise fashion, with 2-acetylthiazole (0.7 eq, 0.57 L). The mixture was carefully heated to 75° C., temperature at which initiation occurred with a strong exothermic event. The mixture was heated to reflux for 5 hours, then cooled to 0-5° C. and aged for 30 minutes. The resulting suspension was filtered and the cake washed with t-butylmethylether. The mother liquors where again cooled to 0-5° C. and the resulting suspension filtered. The cake was washed with t-butylmethylether and the collected solid combined with the previous one to give 704 g of (2E)-3-(dimethylamino)-1-(1,3-thiazol-2-yl)-2-propen-1-one.

The above compound was suspended in ethanol (2.1 L) and cooled down to 10-15° C. A solution of hydrazine hydrate 80% (0.85 L) was added over 15 minutes. The resulting solution was stirred at R.T. for 7 hours, then heated at 60° C. for additional 4 hours and finally cooled again to R.T. The mixture was treated with water, aged for 1 hour then allowed to stand for 16 hours. The resulting suspension was filtered and the solid washed with water. The mother liquors were extracted with ethyl acetate and the organic layer, previously dried on Na₂SO₄, evaporated to solid. The combined solids were slurred in a mixture of ethanol (0.65 L) and dichlomethane (0.25 L), filtered to give 430 g of 2-(1H-pyrazol-3-yl)-1,3-thiazole.

Yield 52% th

The analytical data are according to those reported in WO 03/008412.

6-Methyl-1-[2-methyl-4-(methyloxy)phenyl]-4-[3-(1,3-thiazol-2-yl)-1-H-pyrazol-1-yl]-2,3-dihydro-1H-pyrrolo[2,3-b]pyridine

To a suspension of Cul (2.84 g) in NMP (0.71 L), trans-N,N′-dimethyl-1,2-diaminocyclohexane (31.82 g) was added under a N₂ atmosphere and the green solution stirred at room temperature for 2-12 h (the colour became green-blue). Then potassium carbonate 325 mesh (309.13 g) and 2-(1H-pyrazol-3-yl)-1,3-thiazole (124.01 g) were added followed by the solution of 3-Iodo-6-methyl-1-[2-methyl-4-(methyloxy)phenyl]-2,3-dihydro-1H-pyrrolo[2,3-b]pyridine in NMP (0.71 L). The resulting mixture was heated at 125° C. for 12-18 h. The mixture was then cooled to room temperature and 2 L L of DCM were added followed by about 3 L of water. The combined organic phases were washed twice with water. The organic layer was distilled down to about 1 L (50° C. jacket, 650 mbar), diluted with fresh DCM (1.5 L), again distilled to 0.85 L (50° C. jacket, 650 mbar) and diluted with MeOH (6 L). The methanol solution was heated at 80° C. until complete dissolution was observed, then distilled down to 2.4 L (80° C. jacket, 300 mbar). The solution was brought to room temperature and the resulting suspension was aged for 2 hrs. The title compound was filtered, washed upon the filter with MeOH and dried at 80° C. for 24 h.

Yield 70% th

HPLC greater than 99% a/a

The analytical data are according to those reported in WO 03/008412.

6-methyl-1-[2-methyl-4-(methyloxy)phenyl]-4-[5-(1,3-thiazol-2-yl)-1H-pyrazol-1-yl]-2,3-dihydro-1H-pyrrolo[2,3-b]pyridine

A portion of the crude material was purified by preparative HPLC (column: ABZ plus, 10 cm×21.2 mm, 5 μm; eluting with H₂O/formic acid 0.1%, CH₃CN/formic acid 0.1%, gradient from 0/5% to 0/100%, flow 20 mL/min, DAD and MS⁺ detection; R.T. 3.78 min) to get the title compound as pale yellow solid.

NMR (¹H, CDCl₃): δ 7.87 (d, 1H), 7.77 (d, 1H), 7.40 (d, 1H), 7.18 (d, 1H), 6.93 (d, 1H), 6.82 (d, 1H), 6.78 (dd, 1H), 6.34 (s, 1H), 3.81 (s, 3H), 3.77 (t, 2H), 2.85 (t, 2H), 2.30 (s, 3H), 2.24 (s, 3H).

MS (m/z): 404 [MH]⁺.

Preparation of 6-methyl-1-[2-methyl-6-(methyloxy)-3-pyridinyl]-4-[3-(1,3-thiazol-2-yl)-1H-pyrazol-1-yl]-2,3-dihydro-1H-pyrrolo[2,3-b]pyridine

The title compound may be prepared according to the procedure described just above.

### Example 10

### CRF Binding Activity

CRF binding affinity has been determined in vitro by the compound ability to displace ¹²⁵I-oCRF and ¹²⁵I-Sauvagine for CRF1 and CRF2 SPA, respectively, from recombinant human CRF receptors expressed in Chinese Hamster Ovary (CHO) cell membranes. For membrane preparation, CHO cells from confluent T-flasks were collected in SPA buffer (HEPES/KOH 50 mM, EDTA 2 mM, MgCl₂ 10 mM, pH 7.4.) in 50 mL centrifuge tubes, homogenized with a Polytron and centrifuged (50,000 g for 5 min at 4° C.: Beckman centrifuge with JA20 rotor). The pellet was resuspended, homogenized and centrifuged as before.

The SPA experiment has been carried out in Optiplate by the addition of 100 μL the reagent mixture to 1 μL of compound dilution (100% DMSO solution) per well. The assay mixture was prepared by mixing SPA buffer, WGA SPA beads (2.5 mg/mL), BSA (1 mg/mL) and membranes (50 and 5 μg of protein/mL for CRF1 and CRF2 respectively) and 50 pM of radioligand.

The plate was incubated overnight (>18 hrs) at room temperature and read with the Packard Topcount with a WGA-SPA ¹²⁵I counting protocol.

### Example 11

### CRF Functional Assay

The compound of the invention was characterised in a functional assay for the determination of their inhibitory effect. Human CRF-CHO cells were stimulated with CRF and the receptor activation was evaluated by measuring the accumulation of cAMP.

CHO cells from a confluent T-flask were resuspended with culture medium without G418 and dispensed in a 96-well plate, 25,000 c/well, 100 μL/well and incubated overnight. After the incubation the medium was replaced with 100 μL of cAMP IBMX buffer warmed at 37° C. (5 mM KCl, 5 mM NaHCO₃, 154 mM NaCl, 5 mM HEPES, 2.3 mM CaCl₂, 1 mM MgCl₂, 1 g/L glucose, pH 7.4 additioned by 1 mg/mL BSA and 1 mM IBMX) and 1 μL of antagonist dilution in neat DMSO. After 10 additional minutes of incubation at 37° C. in a plate incubator without CO₂, 1 μL of agonist dilution in neat DMSO was added. As before, the plate was incubated for 10 minutes and then cAMP cellular content was measured by using the Amersham RPA 538 kit.

All publications, including but not limited to patents and patent applications, cited in this specification are herein incorporated by reference as if each individual publication were specifically and individually indicated to be incorporated by reference herein as though fully set forth.

It is to be understood that the present invention covers all combinations of particular and preferred groups described herein above.

The application of which this description and claims forms part may be used as a basis for priority in respect of any subsequent application. The claims of such subsequent application may be directed to any feature or combination of features described herein. They may take the form of product, composition, process, or use claims and may include, by way of example and without limitation, the following claims:

1. A process for preparing compounds of formula (IA) starting from compounds of formula (I) by a coupling reaction catalysed by copper between compounds of formula (I) and a reactive derivative of the upper residue —NR″₂R″₃ 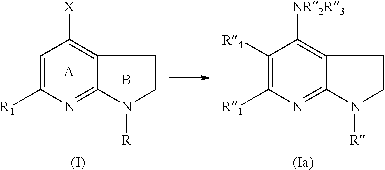
wherein
R is aryl or heteroaryl, each of which may be substituted by 1 to 4 groups selected from:
halogen, C1-C6 alkyl, C1-C6 alkoxy, halo C1-C6 alkyl, C2-C6 alkenyl, C2-C6 alkynyl, halo C1-C6 alkoxy, —C(O)R₅, nitro, —NR₆R₇, cyano, and a group R₈;
R₁ is hydrogen, C1-C6 alkyl, C2-C6 alkenyl, C2-C6 alkynyl, halo C1-C6 alkyl, halo C1-C6 alkoxy, halogen, NR₆R₇ or cyano;
R₅ is C1-C4 alkyl, —OR₆ or —NR₆R₇;
R₆ is hydrogen or C1-C6 alkyl;
R₇ is hydrogen or C1-C6 alkyl;
R₈ is a 5-6 membered heterocycle, which may be saturated or may contain one to three double bonds, and which may be substituted by 1 or more R₁₁ groups;
R₉ is C1-C6 alkyl that may be substituted by one or more groups selected from: C3-C7 cycloalkyl, C1-C6 alkoxy, haloC1-C6 alkoxy, hydroxyl and haloC1-C6 alkyl;
R₁₁ is C3-C7 cycloalkyl, C1-C6 alkyl, C1-C6 alkoxy, halo C1-C6 alkyl, C2-C6 alkenyl, C2-C6 alkynyl, halo C1-C6 alkoxy, hydroxy, halogen, nitro, cyano, or C(O)NR₆R₇;
X is halogen;
R″ corresponds to R;
R″₁ corresponds to R₁;
R₂ is hydrogen, C3-C7 cycloalkyl, or a group R₉;
R₃ is C3-C7 cycloalkyl or a group R₉; or
R₂ and R₃ together with N form a 5-14 membered heterocycle, which may be substituted by 1 to 3 R₁₀ groups;
R″₄ is hydrogen;
R″₅ corresponds to R₅;
R″₆ corresponds to R₆;
R″₇ corresponds to R₇;
R″₈ corresponds to R₈;
R″₉ corresponds to R₉;
R₁₀ is a group R₈, C3-C7 cycloalkyl, C1-C6 alkyl, C1-C6 alkoxy, halo C1-C6 alkyl, C2-C6 alkenyl, C2-C6 alkynyl, halo C1-C6 alkoxy, hydroxy, halogen, nitro, cyano, C(O)NR₆R₇ or phenyl which may be substituted by 1 to 4 R₁₁ groups; and
R″₁₁ corresponds to R₁₁.

2. A process, according to claim 1, for preparing the following compounds:
3-Methyl-4-[6-methyl-4-(3-thiazol-2-yl-pyrazol-1-yl)-2,3-dihydro-1H-pyrrolo[2,3-b]pyridin-1-yl]-benzonitrile;
1-(2,4-Bis-trifluoromethyl-phenyl)-6-methyl-4-(3-thiazol-2-yl-pyrazol-1-yl)-2,3-dihydro-1H-pyrrolo[2,3-b]pyridine;
4-[6-Methyl-4-(3-thiazol-2-yl-pyrazol-1-yl)-2,3-dihydro-1H-pyrrolo[2,3-b]pyridin-1-yl]-3-trifluoromethyl-benzonitrile;
6-Methyl-1-(2-methyl-4-trifluoromethoxy-phenyl)-4-(3-thiazol-2-yl-pyrazol-1-yl)-2,3-dihydro-1H-pyrrolo[2,3-b]pyridine;
1-(4-Methoxy-2-methyl-phenyl)-6-methyl-4-(3-thiazol-2-yl-pyrazol-1-yl)-2,3-dihydro-1H-pyrrolo[2,3-b]pyridine;
1-(2,4-Bis-trifluoromethyl-phenyl)-6-methyl-4-(3-morpholin-4-yl-pyrazol-1-yl)-2,3-dihydro-1H-pyrrolo[2,3-b]pyridine;
1-(2,4-Bis-trifluoromethyl-phenyl)-6-methyl-4-(3-pyridin-2-yl-pyrazol-1-yl)-2,3-dihydro-1H-pyrrolo[2,3-b]pyridine;
4-[1,3′]Bipyrazolyl-1′-yl-1-(2,4-bis-trifluoromethyl-phenyl)-6-methyl-2,3-dihydro-1H-pyrrolo[2,3-b]pyridine.

3. A process for preparing compounds of formula (I) according to the following Scheme 1: 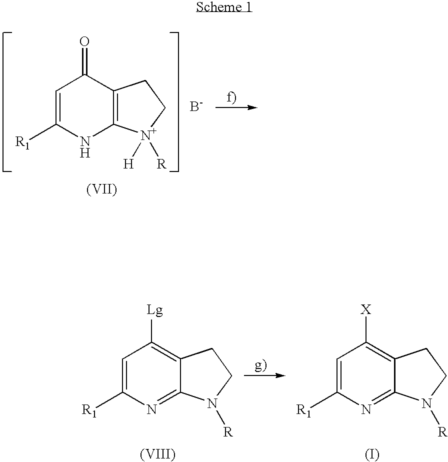
wherein R, R₁, X are defined as in claim 1, and Lg is a leaving group selected among the reactive derivatives of an alkylsulphonic acid;
step f stands for the formation of a reactive derivative of the hydroxy pyridine of compounds (VII); and
step g stands for nucleophilic displacement of the reactive derivative of compounds (VIII) to give the halogenated compounds (I).

4. An intermediate compound of formula (VII) 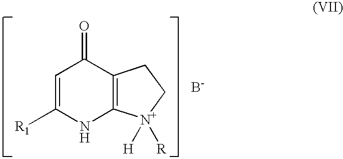
wherein:
R is aryl or heteroaryl each of which may be substituted by 1 to 4 groups selected from:
halogen, C1-C6 alkyl, C1-C6 alkoxy, halo C1-C6 alkyl, C2-C6 alkenyl, C2-C6 alkynyl, halo C1-C6 alkoxy, —C(O)R₅, nitro, —NR₆R₇, cyano, and a group R₈;
R₁ is hydrogen, C1-C6 alkyl, C2-C6 alkenyl, C2-C6 alkynyl, halo C1-C6 alkyl, halo C1-C6 alkoxy, halogen, NR₆R₇ or cyano;
R₅ is C1-C4 alkyl —OR₆ or —NR₆R₇;
R₆ is hydrogen or C1-C6 alkyl;
R₇ is hydrogen or C1-C6 alkyl;
R₈ is a 5-6 membered heterocycle, which may be saturated or may contain one to three double bonds, and which may be substituted by 1 or more R₁₁ groups; and
R₁₁ is C3-C7 cycloalkyl, C1-C6 alkyl, C1-C6 alkoxy, halo C1-C6 alkyl, C2-C6 alkenyl, C2-C6 alkynyl, halo C1-C6 alkoxy, hydroxy, halogen, nitro, cyano, or C(O)NR₆R₇.

5. A process for the preparation of compounds (IV) starting from compounds of formula (II) and comprising the following steps according to Scheme 2: 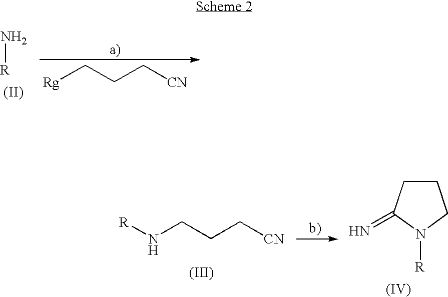
wherein R is defined as in claim 1, Rg is a reactive group selected from: halogen and a reactive derivative of an alkylsulphonic acid;
step a stands for alkylation of the suitable aryl or heteroayl amine of formula (II) with a reactive derivative of butyrronitrile in presence of a base by heating; and
step b stands for the formation of the pyrrolidinone moiety of compounds (IV) which will form the cycle B present in the final compounds (I), by cyclisation of compounds (III), acid catalised and by heating to give the desired compounds (IV).

6. A process for preparing compounds of formula (IVB) according to claim 3 in which step a and step b are performed continuously without isolating intermediate (III), according to the following Scheme 3

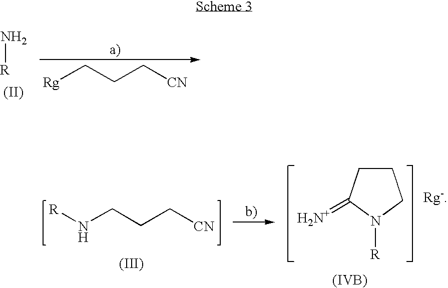

7. An intermediate compound of formula (IVB) 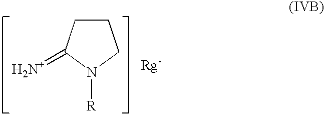
wherein:
R is aryl or heteroaryl each of which may be substituted by 1 to 4 groups selected from:
halogen, C1-C6 alkyl, C1-C6 alkoxy, halo C1-C6 alkyl C2-C6 alkenyl, C2-C6 alkynyl, halo C1-C6 alkoxy, —C(O)R₅, nitro, —NR₆R₇, cyano, and a group R₈;
R₅ is C1-C4 alkyl, —OR₆ or —NR₆R₇;
R₆ is hydrogen or C1-C6 alkyl;
R₇ is hydrogen or C1-C6 alkyl;
R₈ is a 5-6 membered heterocycle, which may be saturated or may contain one to three double bonds, and which may be substituted by 1 or more R₁₁ groups;
R₁₁ is C3-C7 cycloalkyl, C1-C6 alkyl, C1-C6 alkoxy, halo C1-C6 alkyl, C2-C6 alkenyl, C2-C6 alkynyl, halo C1-C6 alkoxy, hydroxy, halogen, nitro, cyano, or C(O)NR₆R₇; and
Rg is a reactive group selected from: halogen and a reactive derivative of an alkylsulphonic acid.

8. A process for the preparation of compounds (VII) starting from compounds of formula (IV) and comprising the following steps: 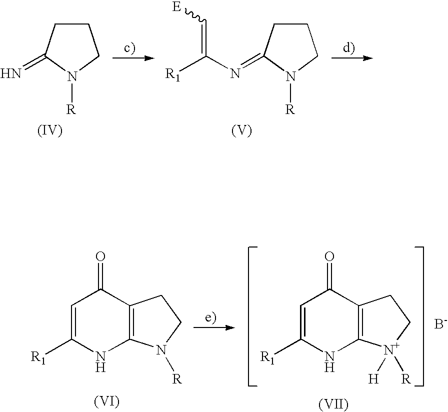
wherein R and R₁, are defined as in claim 1, and
step c stands for a Michael addition of compounds (IV) to a butynoate derivative by heating;
step d stands for cyclisation in basic conditions to give the aromatic compounds (VI); and
step e stands for salt formation by addition of the suitable acid to the compounds (VI).

9. A process for preparing of compounds (VII), according to claim 8, starting from compounds of formula (IV) in which compounds (IV) are replaced by compounds (IVB) according to the following Scheme 5: 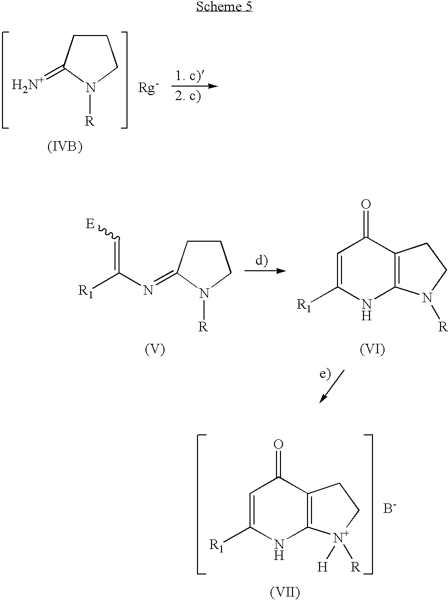
and
step c′ stands for a basic treatment of compounds (IVB) with a suitable base.

10. Compound of formula (IX) or a pharmaceutically acceptable salt thereof.

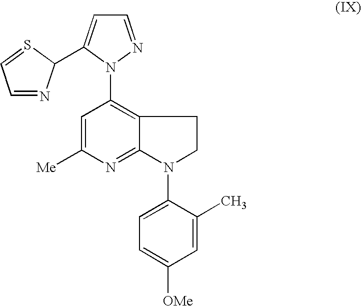

11-16. (canceled)

17. A pharmaceutical composition comprising the compound according to claim 10 or a pharmaceutically acceptable salt thereof in admixture with one or more physiologically acceptable carriers or excipients.

18. A method for the treatment of a condition mediated by CRF (corticotropin-releasing factor), comprising administration of an effective amount of a compound according to claim 10 or a pharmaceutically acceptable salt thereof to a mammal in need of treatment thereof.

19. A method, according to claim 17, wherein the condition mediated by CRF is depression or anxiety.

20. A method, according to claim 17, wherein the condition mediated by CRF is IBS (irritable bowel disease) or IBD (inflammatory bowel disease.
